(19) United States
(12) Patent Application Publication
(10) Pub. No.: US 2025/0127471 A1
(43) Pub. Date: Apr. 24, 2025

(54) MAMMOGRAPHY APPARATUS, DISPLAY METHOD OF MAMMOGRAPHY APPARATUS, AND DISPLAY PROGRAM OF MAMMOGRAPHY APPARATUS

(71) Applicant: FUJIFILM Corporation, Tokyo (JP)

(72) Inventors: Hisatsugu HORIUCHI, Kanagawa (JP); Atsushi OSAWA, Kanagawa (JP); Rika TASHIRO, Kanagawa (JP); Takeyasu KOBAYASHI, Kanagawa (JP); Sayaka SAITO, Kanagawa (JP)

(73) Assignee: FUJIFILM Corporation, Tokyo (JP)

(21) Appl. No.: 18/915,255

(22) Filed: Oct. 14, 2024

(30) Foreign Application Priority Data
Oct. 18, 2023 (JP) 2023-179898

(51) Int. Cl. A61B 6/50; A61B 6/00; A61B 6/04; A61B 8/00; A61B 8/08

(52) U.S. Cl. CPC A61B 6/502; A61B 6/4417; A61B 6/488; A61B 6/547; A61B 8/0825; A61B 8/464; A61B 6/0414

(57) ABSTRACT

A first display unit that is provided at a position of an arm part in front of an examinee, and a second display unit that is provided at a side surface of the arm part are provided. Then, control of switching between the display units that display an ultrasound image is performed according to a rotational position of the arm part.

### CROSS-REFERENCE TO RELATED APPLICATIONS

This application claims priority under 35 USC 119 from Japanese Patent Application No. 2023-179898 filed on Oct. 18, 2023, the disclosure of which is incorporated by reference herein.

### BACKGROUND

### 1. Technical Field

The present disclosure relates to a mammography apparatus, a display method of the mammography apparatus, and a display program of the mammography apparatus.

### 2. Related Art

JP2012-29785A has proposed a radiation image capturing method of irradiating a subject with radiation from a plurality of different imaging directions by moving a radiation source and capturing a radiation image for each imaging direction by the irradiation with the radiation, in which images are acquired by a modality that acquires images related to biological information of the subject other than radiation images while the radiation source is moved from a predetermined imaging direction to the next imaging direction.

JP2009-82402A has proposed a medical image diagnosis system including a medical imaging apparatus that acquires a radiation image of an object projected onto a projection plane by irradiating the object with radiation, acquires an ultrasound image of the object by transmitting and receiving ultrasound waves to and from the object, and generates first image data representing an ultrasound slice image along a slice plane substantially orthogonal to the projection plane, second image data representing the radiation image, and position data representing a position of the slice plane in the projection plane; a medical image storage apparatus that stores the first image data, the second image data, and the position data in association with each other; and a medical image display apparatus that displays the ultrasound slice image on the basis of the first image data and displays the radiation image in which a marker representing the position of the slice plane in the projection plane is represented, on the basis of the second image data and the position data.

JP2022-57943A has proposed an information processing apparatus in which, in a mammography apparatus that captures a radiation image by irradiating, with radiation, a breast in a compressed state by a compression member disposed between a radiation source and a radiation detector, an image projection unit that projects a projection image onto a first projection plane of the compression member is controlled to switchably project at least one of first information or second information onto the first projection plane.

JP2016-538075A has proposed a mammography apparatus including a certain basic structural portion and a user interface configuration disposed in the basic structural portion, in which the user interface configuration can adjust the orientation of a view of the user interface to either an orientation in which the view can be visually recognized only by an operator of the apparatus or an orientation in which the view can be visually recognized by a patient positioned for imaging.

### SUMMARY

In a mammography apparatus including a support portion that rotatably supports a radiation source and a display unit that displays an ultrasound image, when the ultrasound image is displayed, it may be difficult to see the ultrasound image on the display unit depending on a rotational position of the support portion.

The present disclosure is made in consideration of the circumstances, and an object thereof is to provide a mammography apparatus, a display method of the mammography apparatus, and a display program of the mammography apparatus which can maintain visibility of a display unit even in a case where a support portion is rotated in the mammography apparatus including the support portion that rotatably supports a radiation source and the display unit that displays an ultrasound image.

In order to achieve the object, a mammography apparatus according to a first aspect of the present disclosure includes a support portion that rotatably supports a radiation source that emits radiation, at a plurality of imaging positions where incidence angles of the radiation to a breast of an examinee are different from each other; a plurality of display units that display an ultrasound image of the breast acquired by an ultrasound probe, and are provided at different positions; and at least one processor, in which the processor performs processing of displaying the ultrasound image by switching between the plurality of display units according to a rotational position of the support portion.

In a mammography apparatus according to a second aspect of the present disclosure, in the mammography apparatus according to the first aspect, in a case of switching between the plurality of display units according to the rotational position, in a case where there are the plurality of display units that can be visually recognized by an operator, the processor displays the ultrasound image by preferentially switching to the display unit close to the breast.

In a mammography apparatus according to a third aspect of the present disclosure, in the mammography apparatus according to the first aspect, the plurality of display units are direct-viewing electronic displays.

In a mammography apparatus according to a fourth aspect of the present disclosure, in the mammography apparatus according to the first aspect, the plurality of display units are projection-type electronic displays which display a projection image from a projection unit.

In a mammography apparatus according to a fifth aspect of the present disclosure, in the mammography apparatus according to the fourth aspect, a support table that supports the support portion is provided with a reflecting surface functioning as the projection-type electronic display.

In a mammography apparatus according to a sixth aspect of the present disclosure, in the mammography apparatus according to the fourth aspect, the projection unit has a reflective member, and switches between projection positions of the electronic display by changing at least one of an angle or a position of the reflective member.

In a mammography apparatus according to a seventh aspect of the present disclosure, in the mammography apparatus according to the sixth aspect, a plurality of reflective members are provided.

In the mammography apparatus according to the first aspect, a mammography apparatus according to an eighth aspect of the present disclosure further includes a detection unit that detects a rotation angle of the support portion, in which in a case where the rotation angle equal to or greater than a preset threshold value is detected by the detection unit, the processor displays the ultrasound image by switching between the plurality of display units.

In the mammography apparatus according to the first aspect, a mammography apparatus according to a ninth aspect of the present disclosure further includes a detection unit that detects a rotation angle of the support portion, in which the processor displays the ultrasound image by switching to a predetermined display unit according to an angle detected by the detection unit, among the plurality of display units.

A display method of a mammography apparatus according to a tenth aspect of the present disclosure includes, via a computer, performing processing of displaying an ultrasound image of a breast acquired by an ultrasound probe, by switching between a plurality of display units, which display the ultrasound image and are provided at different positions, according to a rotational position of a support portion that rotatably supports a radiation source that emits radiation, at a plurality of imaging positions where incidence angles of the radiation to the breast of an examinee are different from each other.

A display program of a mammography apparatus according to an eleventh aspect of the present disclosure causes a computer to execute processing of displaying an ultrasound image of a breast acquired by an ultrasound probe, by switching between a plurality of display units, which display the ultrasound image and are provided at different positions, according to a rotational position of a support portion that rotatably supports a radiation source that emits radiation, at a plurality of imaging positions where incidence angles of the radiation to the breast of an examinee are different from each other.

According to the present disclosure, in a mammography apparatus including a support portion that rotatably supports a radiation source and a display unit that displays an ultrasound image, it is possible to maintain visibility of a display unit even in a case where the support portion is rotated.

### BRIEF DESCRIPTION OF THE DRAWINGS

Exemplary embodiments of the technology of the disclosure will be described in detail based on the following figures, wherein:

FIG. 1 is a configuration diagram schematically illustrating an example of an overall configuration of a mammography system of the present embodiment;

FIG. 2 is a side view illustrating an example of the appearance of a mammography apparatus of the present embodiment;

FIG. 3 is a diagram illustrating an example of a compression member of the present embodiment;

FIG. 4 is a diagram illustrating an example of a moving mechanism of an ultrasound probe of the present embodiment;

FIG. 5 is a block diagram illustrating an example of configurations of the mammography apparatus and a console of the present embodiment;

FIG. 6 is a diagram illustrating a state in which an angle of an arm part of the mammography apparatus of the present embodiment is 0 degrees;

FIG. 7 is a diagram illustrating a state in which an angle of the arm part of the mammography apparatus of the present embodiment is 90 degrees;

FIG. 8 is a flowchart illustrating an example of a flow of processing performed by a controller of the mammography apparatus in the mammography system according to the present embodiment;

FIG. 9 is a diagram for describing an example of preferentially switching to a display unit close to the breast in a case where the angle of the arm part is 0 degrees;

FIG. 10 is a diagram for describing an example of preferentially switching to a display unit close to the breast in a case where the angle of the arm part is 90 degrees;

FIG. 11 is a diagram illustrating an example of a projection unit and a first reflecting surface in a case where a projection-type electronic display is applied;

FIG. 12 is a diagram of the arm part as viewed from above;

FIG. 13 is a diagram of the arm part as viewed from the front;

FIG. 14 is a diagram illustrating a state in which a projection image is projected onto from the projection unit to a second reflecting surface in a state where the angle of the arm part of the mammography apparatus of the present embodiment is 90 degrees;

FIG. 15 is a diagram illustrating the switching of projection positions by rotation of a reflective member;

FIG. 16 is a diagram illustrating the switching of projection positions by movement of an optical unit;

FIG. 17 is a diagram illustrating another arrangement example of the projection unit, the first reflecting surface, and the second reflecting surface; and

FIG. 18 is a diagram illustrating an example of the mammography apparatus in which a biopsy unit and the moving mechanism are omitted.

### DETAILED DESCRIPTION

Hereinafter, embodiments of the present invention will be described in detail with reference to the drawings. Note that the present embodiment does not limit the present invention.

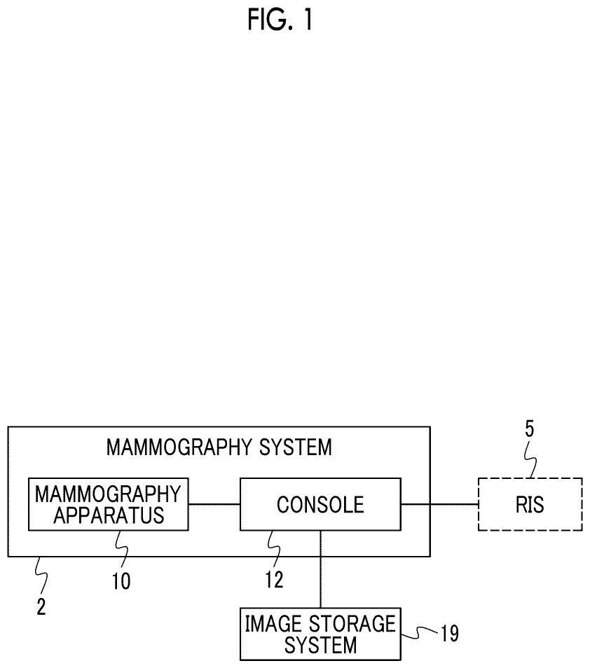

First, an outline of an example of the overall configuration of a mammography system 2 of the present embodiment will be described. FIG. 1 illustrates a configuration diagram schematically illustrating an example of the overall configuration of the mammography system 2 of the present embodiment. As illustrated in FIG. 1, the mammography system 2 includes a mammography apparatus 10 and a console 12.

The mammography apparatus 10 is an apparatus capable of capturing a radiation image and an ultrasound image of a breast of an examinee and capable of performing a biopsy of the breast, using the breast of the examinee as a subject. For example, the mammography apparatus 10 of the present embodiment is an apparatus to which a function of performing ultrasonography is added to a mammography apparatus capable of performing a biopsy.

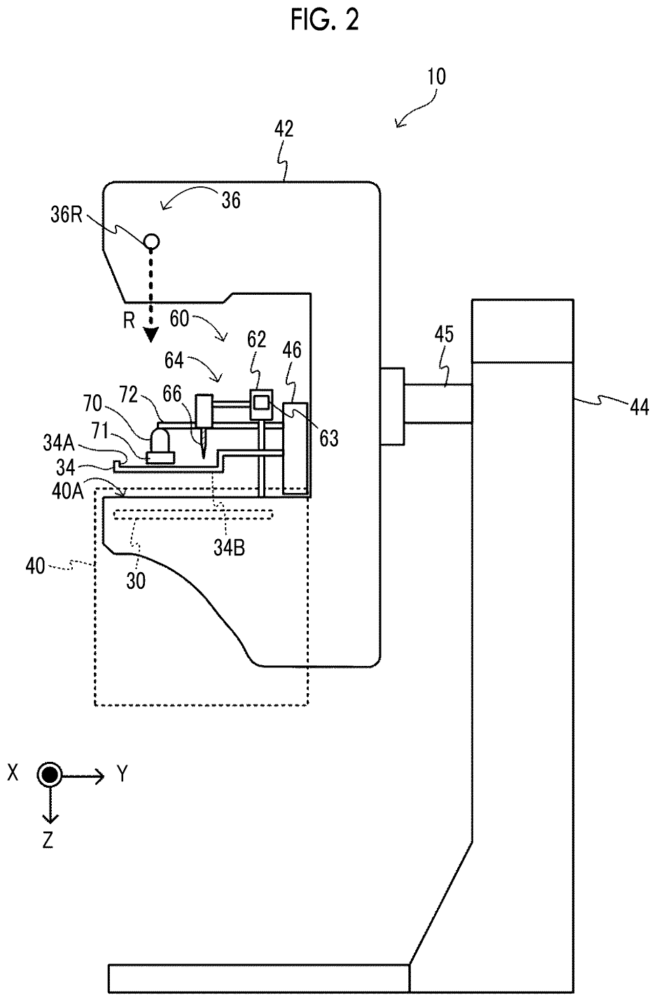

FIG. 2 is a side view illustrating an example of the appearance of the mammography apparatus 10 of the present embodiment. In addition, FIG. 2 is a side view illustrating the mammography apparatus 10 as viewed from the right side of an examinee. As illustrated in FIG. 2, the mammography apparatus 10 includes a radiation source 36R, a radiation detector 30, an imaging table 40 disposed between the radiation source 36R and the radiation detector 30, and a compression member 34 that compresses the breast between the compression member 34 and the imaging table 40.

The radiation detector 30 is disposed inside the imaging table 40. The radiation detector 30 detects radiation R transmitted through the breast as the subject. In the mammography apparatus 10 of the present embodiment, in a case where imaging is performed, the breast of the examinee is positioned on an imaging surface 40A of the imaging table 40 by an operator such as a doctor or a radiology technician. The imaging surface 40A or the like with which the breast of the examinee comes into contact is formed of carbon or the like in terms of the transmittance and intensity of the radiation R, for example.

The radiation detector 30 detects the radiation R transmitted through the breast of the examinee and the imaging table 40, generates a radiation image on the basis of the detected radiation R, and outputs the generated radiation image. The type of the radiation detector 30 of the present embodiment is not particularly limited, and for example, the radiation detector 30 may be an indirect conversion type radiation detector that converts the radiation R into light and converts the converted light into electric charges, or may be a direct conversion type radiation detector that directly converts the radiation R into electric charges.

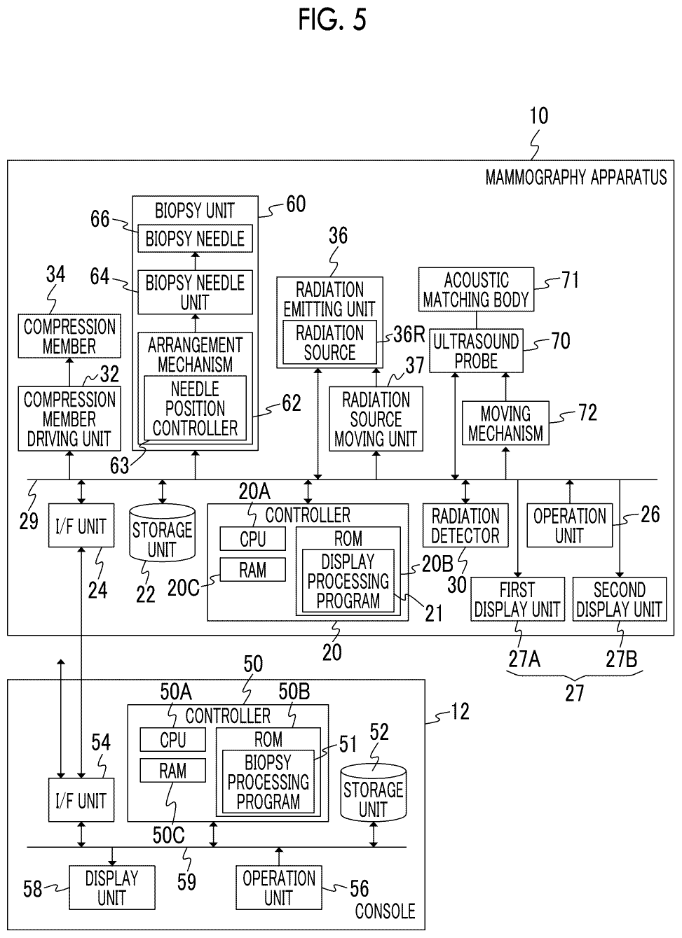

The radiation source 36R is provided in a radiation emitting unit 36. As illustrated in FIG. 2, the radiation emitting unit 36 is provided in an arm part 42 as an example of a support portion (bracket), together with the imaging table 40 and a compression unit 46. Note that, as illustrated in FIG. 2, the mammography apparatus 10 of the present embodiment includes the arm part 42, a base 44 as an example of a support table, and a shaft portion 45. The arm part 42 is held by the base 44 to be movable in an up-down direction (Z-axis direction). The shaft portion 45 connects the arm part 42 to the base 44. In addition, the arm part 42 rotatably supports the radiation source 36R at a plurality of imaging positions where incidence angles of the radiation to the breast of the examinee are different from each other. Specifically, the arm part 42 is rotatable relative to the base 44 by a radiation source moving unit 37 (refer to FIG. 5) with the shaft portion 45 as a rotation axis. By rotating the arm part 42 relative to the base 44, the incidence angle of the radiation to the breast placed on the imaging surface 40A of the imaging table 40 can be varied. As described above, the mammography apparatus 10 according to the present embodiment can perform so-called stereo imaging or tomosynthesis imaging. Note that the radiation source moving unit 37 functions as an example of a detection unit (detector), and has a function of detecting the rotational position of the arm part 42. For example, a sensor that detects the rotational position of the arm part 42 may be included, or the rotational position may be detected by detecting a drive amount of the radiation source moving unit 37.

In addition, as illustrated in FIG. 2, the compression member 34 is attached to the compression unit 46. The compression unit 46 and the arm part 42 can be relatively rotated with respect to the base 44 separately, using the shaft portion 45 as the rotation axis. In the present embodiment, gears (not illustrated) are provided in each of the shaft portion 45, the arm part 42, and the compression unit 46. Each gear is switched between an engaged state and a disengaged state to connect each of the arm part 42 and the compression unit 46 to the shaft portion 45. One or both of the arm part 42 and the compression unit 46 connected to the shaft portion 45 are rotated integrally with the shaft portion 45.

The compression member 34 of the present embodiment is a plate-shaped member, and is moved in the up-down direction (Z-axis direction) by a compression member driving unit 32 (refer to FIG. 5) provided in the compression unit 46 to compress the breast of the examinee between the compression member 34 and the imaging table 40.

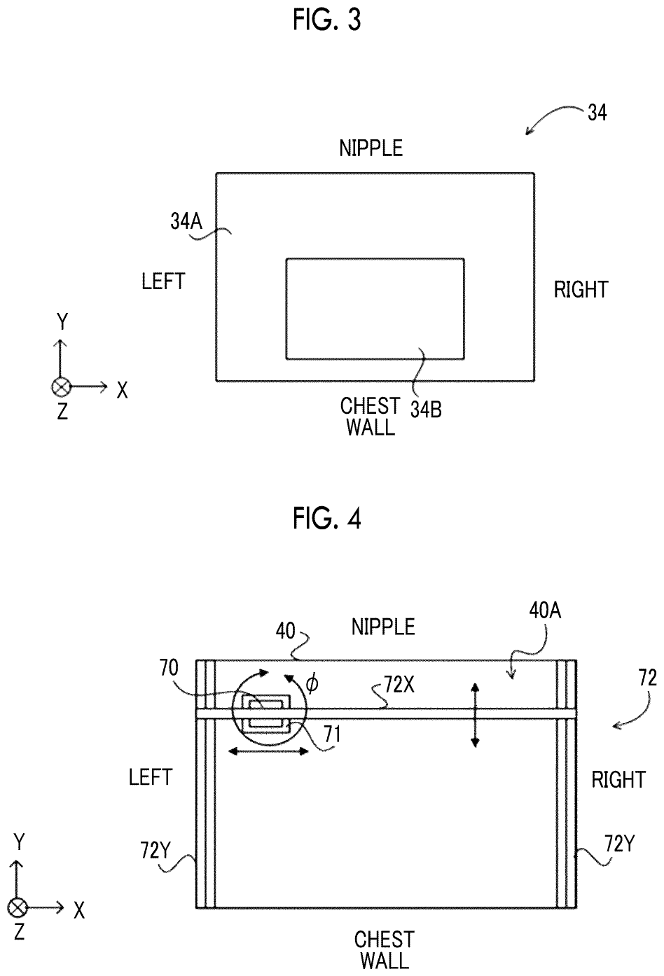

It is preferable that the compression member 34 is optically transparent in order to check positioning or a compression state in the compression of the breast. In addition, the compression member 34 is formed of a material having excellent transmittance of the radiation R. Furthermore, the compression member 34 of the present embodiment is a compression member for a biopsy, and as illustrated in FIG. 3, the compression member 34 has an opening portion 34B at a bottom portion 34A that is in contact with the breast in a case of compressing the breast. A biopsy needle 66 and an ultrasound probe 70 can be inserted into the opening portion 34B.

In addition, as illustrated in FIG. 2, the mammography apparatus 10 according to the present embodiment includes a biopsy unit 60. The biopsy unit 60 includes an arrangement mechanism 62, the biopsy needle 66, and a biopsy needle unit 64. The biopsy needle 66 is provided in the biopsy needle unit 64, and is used to puncture the breast and collect tissues. The arrangement mechanism 62 has a function of moving the biopsy needle 66 in X, Y, and Z directions to arrange the biopsy needle 66 at a target insertion position. Note that, in the present embodiment, the X direction is a left-right direction of the examinee, and the Y direction is a front-rear direction of the examinee. In addition, the Z direction is a direction perpendicular to the XY plane, and is the up-down direction of the examinee. Therefore, hereinafter, the X direction is referred to as the “left-right direction”, the Y direction is referred to as the “front-rear direction”, and the Z direction is referred to as the “up-down direction”.

The arrangement mechanism 62 includes a needle position controller 63, and a position of a distal end of the biopsy needle 66 is controlled by the needle position controller 63. In a case where position information of a target is received, the needle position controller 63 moves the position of the distal end of the biopsy needle 66 to a target insertion position corresponding to the position of the target, and inclines the biopsy needle 66 according to a target insertion angle. The biopsy needle 66 arranged by the arrangement mechanism 62 in this manner is made to puncture the breast, and the collection of the target, that is, a biopsy is performed by the biopsy needle 66.

In addition, as illustrated in FIG. 2, the mammography apparatus 10 of the present embodiment includes the ultrasound probe 70 to which an acoustic matching body 71 is attached, and a moving mechanism 72.

The ultrasound probe 70 is used for acquiring an ultrasound image of the breast by scanning the breast with ultrasound waves. The ultrasound probe 70 includes a plurality of ultrasound transducers (not illustrated) which are one-dimensionally or two-dimensionally arranged. Each of the ultrasound transducers transmits ultrasound waves on the basis of an applied drive signal, receives ultrasound echoes, and outputs a reception signal. The acoustic matching body 71 is provided in the ultrasound probe 70 to cover at least a transmission surface for the ultrasound waves, which comes into contact with the breast. The acoustic matching body 71 is formed of a member having an acoustic impedance close to that of a living body (breast), and is in the form of a gel or a sheet in the present embodiment. As such an acoustic matching body 71, an acoustic coupler or the like can be used. Specific examples thereof include an acoustic coupler of a type in which a polyurethane gel pad is fixed to the ultrasound probe 70 with a fixing jig.

The moving mechanism 72 has a function of moving the ultrasound probe 70 in the front-rear, left-right, and up-down directions. As illustrated in FIG. 4, the moving mechanism 72 includes one moving rail 72X and two moving rails 72Y. The moving rail 72X is a rail extending along the left-right direction, and the ultrasound probe 70 is attached to the moving rail 72X. The moving mechanism 72 changes the position of the ultrasound probe 70 along the left-right direction by moving the ultrasound probe 70 along the moving rail 72X.

In addition, the moving rail 72Y is a rail provided at each of both ends of the imaging surface 40A of the imaging table 40 and extending along the front-rear direction. The moving mechanism 72 changes the position of the ultrasound probe 70 along the front-rear direction by moving the moving rail 72X to which the ultrasound probe 70 is attached, along the front-rear direction.

In addition, the moving mechanism 72 can also change the height of the ultrasound probe 70 by moving the moving rails 72X and 72Y in the up-down direction.

Furthermore, in the present embodiment, as illustrated in FIG. 4, the moving mechanism 72 can change the breast contact orientation by rotating the ultrasound probe 70 (rotation angle q).

On the other hand, the console 12 of the present embodiment has a function of controlling the mammography apparatus 10 using an imaging order and various kinds of information acquired from a radiology information system (RIS) 5 or the like via a wireless communication local area network (LAN), instructions input by a user using an operation unit 56 (refer to FIG. 5) or the like, and the like. In addition, the console 12 is also connected to an image storage system 19 through wireless communication or wired communication, and has a function of transmitting radiation images and ultrasound images obtained by the mammography apparatus 10 to the image storage system 19 such as picture archiving and communication systems (PACS) and storing the images.

Furthermore, configurations of the mammography apparatus 10 and the console 12 will be described with reference to FIG. 5. FIG. 5 illustrates a block diagram illustrating an example of the configurations of the mammography apparatus 10 and the console 12.

As illustrated in FIG. 5, the mammography apparatus 10 further includes a controller 20, a storage unit 22, an I/F unit 24, an operation unit 26, and a display unit (first display unit 27A and second display unit 27B) 27. The controller 20, the storage unit 22, the I/F unit 24, the operation unit 26, the first display unit (display) 27A, the second display unit (display) 27B, the radiation detector 30, the compression member driving unit 32, the radiation emitting unit 36, the radiation source moving unit 37, the biopsy unit 60, the ultrasound probe 70, and the moving mechanism 72 are connected to each other to be able to transmit and receive various kinds of information, via a bus 29 such as a system bus or a control bus.

The controller 20 of the present embodiment controls the overall operation of the mammography apparatus 10. The controller 20 includes a central processing unit (CPU) 20A, a read only memory (ROM) 20B, and a random access memory (RAM) 20C. Various programs and the like executed by the CPU 20A, such as an imaging processing program and a display processing program 21 executed in the case of controlling imaging, are stored in advance in the ROM 20B. The RAM 20C temporarily stores various kinds of data.

The storage unit 22 stores radiation images captured using the radiation detector 30, ultrasound images captured using the ultrasound probe 70, various other kinds of information, and the like. Specific examples of the storage unit 22 include an HDD, an SSD, and the like.

The operation unit 26 is used by the user to input instructions, various kinds of information, and the like regarding the imaging and biopsy. Note that the operation unit 26 is not particularly limited, and examples of the operation unit 26 include various switches, a touch panel, a touch pen, a mouse, and the like.

The first display unit 27A and the second display unit 27B are provided at different positions, respectively, and display an ultrasound image of the breast of the examinee acquired by the ultrasound probe. In the present embodiment, as illustrated in FIG. 6, the first display unit 27A is provided at a position of the arm part 42 in front of the examinee, and the second display unit 27B is provided on a side surface of the arm part 42. In the present embodiment, the ultrasound image is displayed by switching between the first display unit 27A and the second display unit 27B according to the rotational position of the arm part 42 with respect to the base 44. Note that the first display unit 27A and the second display unit 27B may also display other images such as radiation images other than the ultrasound images. In addition, the second display unit 27B may be provided on one side surface of the arm part 42 or on both side surfaces of the arm part 42. In addition, as an example, a direct-viewing type electronic display is applied to the first display unit 27A and the second display unit 27B. In addition, a space for providing the display unit 27 is limited, and it is considered that providing the display unit 27 to the arm part 42 is particularly effective in order for the user to check the ultrasound image without significantly moving the line of sight from an actual observation target (breast). However, in that case, there is a concern that the display will be difficult to see depending on the rotational position of the arm part 42, and it is suitable that the display position is changed according to the rotational position as in the present embodiment even in such a case.

The I/F unit 24 performs communication of radiation images, ultrasound images, and various kinds of information with the console 12 via wireless communication or wired communication.

On the other hand, as illustrated in FIG. 5, the console 12 includes a controller 50, a storage unit 52, an I/F unit 54, the operation unit 56, and a display unit 58. The controller 50, the storage unit 52, the I/F unit 54, the operation unit 56, and the display unit 58 are connected to each other to be able to transmit and receive various kinds of information, via a bus 59 such as a system bus or a control bus.

The controller 50 of the present embodiment controls the overall operation of the console 12. The controller 50 includes a CPU 50A, a ROM 50B, and a RAM 50C. Various programs and the like executed by the CPU 50A, such as a biopsy processing program 51 executed in the case of controlling a biopsy, are stored in the ROM 50B in advance. The RAM 50C transitorily stores various kinds of data.

The storage unit 52 stores radiation images and ultrasound images captured using the mammography apparatus 10, various other kinds of information, and the like. Specific examples of the storage unit 52 include an HDD, an SSD, and the like.

The operation unit 56 is used by the user to input instructions, various kinds of information, and the like regarding the imaging and biopsy. Note that the operation unit 56 is not particularly limited, and examples of the operation unit 56 include various switches, a touch panel, a touch pen, a mouse, and the like. The display unit 58 displays various kinds of information. Note that the operation unit 56 and the display unit 58 may be integrated into a touch panel display.

The I/F unit 54 performs communication of radiation images, ultrasound images, and various kinds of information with each of the RIS 5, the mammography apparatus 10, and the image storage system 19 via wireless communication or wired communication.

Meanwhile, in the mammography apparatus 10 configured as described above, in a case of checking the ultrasound image while checking the position of the ultrasound probe 70, in a case where the arm part 42 is rotated, the ultrasound image may be difficult to see depending on the position of the display unit 27 that displays the ultrasound image.

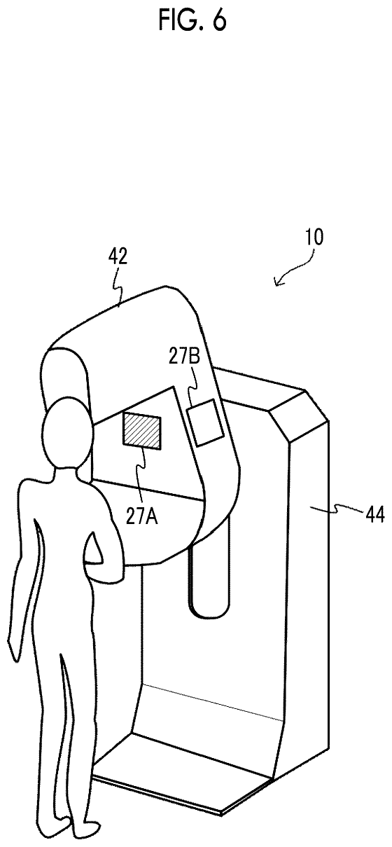

Therefore, in the mammography apparatus 10 according to the present embodiment, a plurality of display units (two display units of the first display unit 27A and the second display unit 27B in the present embodiment) 27 are provided at different positions, respectively. Specifically, as illustrated in FIGS. 6 and 7, the first display unit 27A provided at a position of the arm part 42 in front of the examinee and the second display unit 27B provided on the side surface of the arm part 42 are provided. Then, the control of switching between the display units that display the ultrasound image is performed according to the rotational position of the arm part 42. For example, in a case of CC imaging (the angle of the arm part 42 is 0 degrees), the ultrasound image is displayed on the first display unit 27A as illustrated in FIG. 6. In addition, in a case of MO imaging (the angle of the arm part 42 is 90 degrees), the ultrasound image is displayed on the second display unit 27B as illustrated in FIG. 7. In addition, in a case of MLO imaging (the angle of the arm part 42 is 50 to 70 degrees), the ultrasound image is displayed on the second display unit 27B. In other words, in a case where the rotational position of the arm part 42 is 50 degrees or more, the display is switched to the second display unit 27B, and in a case where the rotational position of the arm part 42 is greater than-50 degrees and less than 50 degrees, the display is switched to the first display unit 27A. That is, in a case where the rotation angle of the arm part 42 equal to or greater than a preset threshold value is detected, the plurality of display units 27 are switched to display the ultrasound image, and the ultrasound image is displayed by switching to the predetermined display unit 27, among the plurality of display units, according to the rotation angle of the arm part 42.

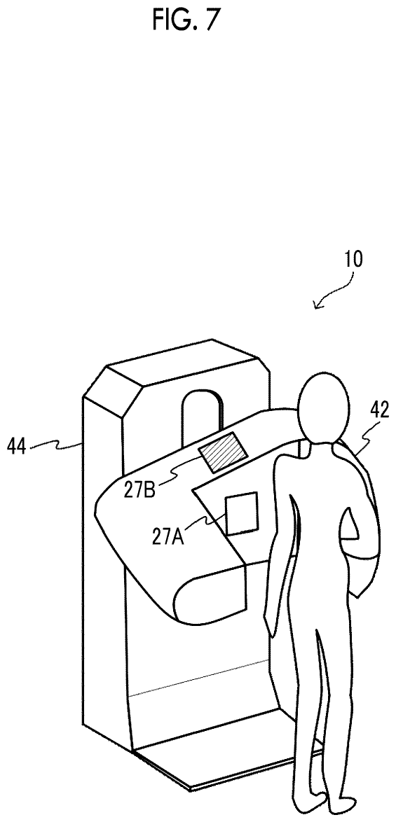

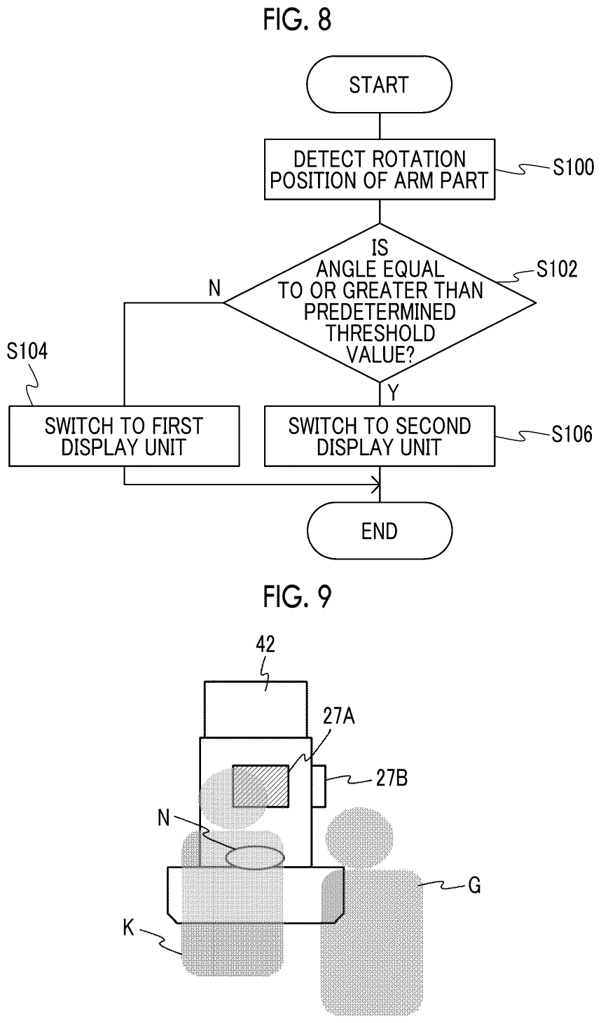

Subsequently, specific processing performed by the controller 20 of the mammography apparatus 10 configured as described above will be described. FIG. 8 is a flowchart illustrating an example of a flow of processing performed by the controller 20 of the mammography apparatus 10 in the mammography system 2 according to the present embodiment. Note that the processing in FIG. 8 is performed by the CPU 20A executing the display processing program 21, and is started, for example, in a case where the imaging of the ultrasound image is instructed.

In step S100, the CPU 20A detects the rotational position of the arm part 42, and the processing transitions to step S102. For example, the rotational position of the arm part 42 is detected by a function of the radiation source moving unit 37 which detects the rotational position of the arm part 42.

In step S102, the CPU 20A determines whether or not the absolute value of the detected rotation angle of the arm part 42 is equal to or greater than a predetermined threshold value. For example, the determination is made as to whether or not the angle is 50 degrees or more or −50 degrees or less in the MLO imaging. In a case where the determination result is negative, the processing transitions to step S104, and in a case where the determination result is affirmative, the processing transitions to step S106.

In step S104, the CPU 20A switches the display of the ultrasound image to the first display unit 27A, and the series of processing is ended. That is, in a case where the rotation angle of the arm part 42 is the CC imaging illustrated in FIG. 6, the ultrasound image is displayed on the first display unit 27A at the position of the arm part 42 in front of the examinee. As a result, the operator such as a doctor or a radiology technician can check the ultrasound image using the first display unit 27A that is at a position where the ultrasound image is easy to see while checking the position of the ultrasound probe 70.

On the other hand, in step S106, the CPU 20A switches the display of the ultrasound image to the second display unit 27B, and the series of processing is ended. That is, in a case where the rotation angle of the arm part 42 is the MO imaging or the MLO imaging illustrated in FIG. 7, the ultrasound image is displayed on the second display unit 27B provided on the side surface of the arm part 42. As a result, the operator can check the ultrasound image using the second display unit 27B that is at a position where the ultrasound image is easier to see than the first display unit 27A, while checking the position of the ultrasound probe 70.

In the processing in FIG. 8, the rotation angle of the arm part 42 is detected and the first display unit 27A and the second display unit 27B are switched, but the first display unit 27A and the second display unit 27B may be switched on the basis of the content of the imaging instruction. That is, the imaging instruction corresponding to the rotation angle may be checked in step S100, and, it may be determined in step S102 whether or not the imaging instruction is the MO imaging or the MLO imaging.

Note that, in the embodiment described above, the case where the two display units of the first display unit 27A and the second display unit 27B are provided has been described, but three or more display units may be provided. In this case, in a case of switching between the plurality of display units according to the rotational position of the arm part 42, in a case where there are the plurality of display units that can be visually recognized by the operator, the ultrasound image may be displayed by preferentially switching to the display unit close to the breast. For example, in a case where a right breast N of a patient is observed in a positional relationship of FIG. 9, since a radiology technician G stands on the right side of a patient K, the first display unit 27A is close to the breast position between the first display unit 27A and the second display unit 27B. Therefore, among these, displaying the image on the display unit closer to the breast N among the display units, that is, on the first display unit 27A is suitable because the radiology technician G can check the ultrasound image without significantly moving the line of sight after checking the breast N as the actual observation target. In addition, in a case where the right breast N of the patient K is observed in a positional relationship of FIG. 10, since the radiology technician G stands on the left side of the patient K, the second display unit 27B is close to the breast position between the first display unit 27A and the second display unit 27B. Therefore, among these, displaying the image on the display unit closer to the breast N among the display units, that is, on the second display unit 27B is suitable because the radiology technician G can check the ultrasound image without significantly moving the line of sight after checking the breast N as the actual observation target.

In addition, in the embodiment described above, an example in which a direct-viewing type electronic display is applied as an example of the first display unit 27A and the second display unit 27B has been described, but a projection-type electronic display that displays a projection image from a projection unit may be applied as the first display unit 27A and the second display unit 27B.

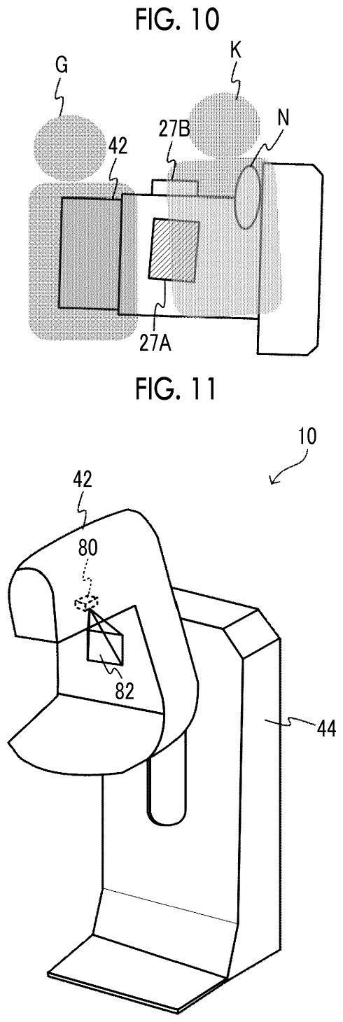

In a case where a projection-type electronic display is applied, for example, as illustrated in FIG. 11, a projection unit (projector) 80 that displays the projection image is provided in the vicinity of the radiation source 36R on the upper portion of the arm part 42, and a first reflecting surface 82 as a projected surface is disposed at a position in front of the examinee. Accordingly, by projecting the ultrasound image from the projection unit 80 to the first reflecting surface 82, the ultrasound image can be displayed at the same position as the first display unit 27A.

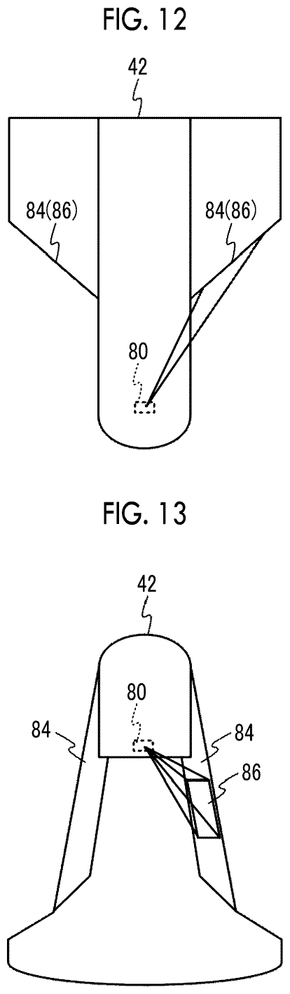

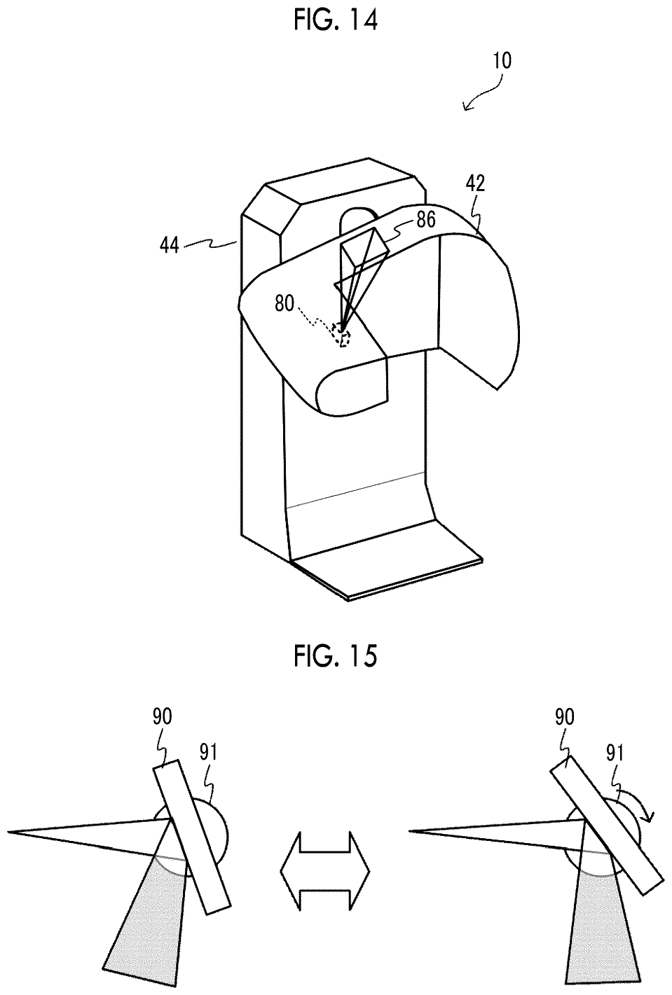

In addition, as illustrated in the top view of the arm part 42 in FIG. 12 and the front view of the arm part 42 in FIG. 13, an inclined surface 84 that is inclined toward the examinee is provided on the side surface of the arm part 42, and a second reflecting surface 86 as the projected surface is disposed on the inclined surface 84. Accordingly, by projecting the ultrasound image from the projection unit 80 to the second reflecting surface 86, the ultrasound image can be displayed at the same position as the second display unit 27B, as illustrated in FIG. 14. Note that the second reflecting surface 86 may be provided on both side surfaces of the arm part 42. In addition, the first reflecting surface 82 and the second reflecting surface 86 have a planar shape, and it is suitable that the surfaces thereof are diffusely reflecting surfaces (for example, surfaces subjected to textured processing).

In addition, in the switching between the first reflecting surface 82 and the second reflecting surface 86, the projection positions may be switched by, for example, rotating a reflective member 90, such as a rotatable optical mirror, by driving a driving unit 91 including a motor or the like as illustrated in FIG. 15.

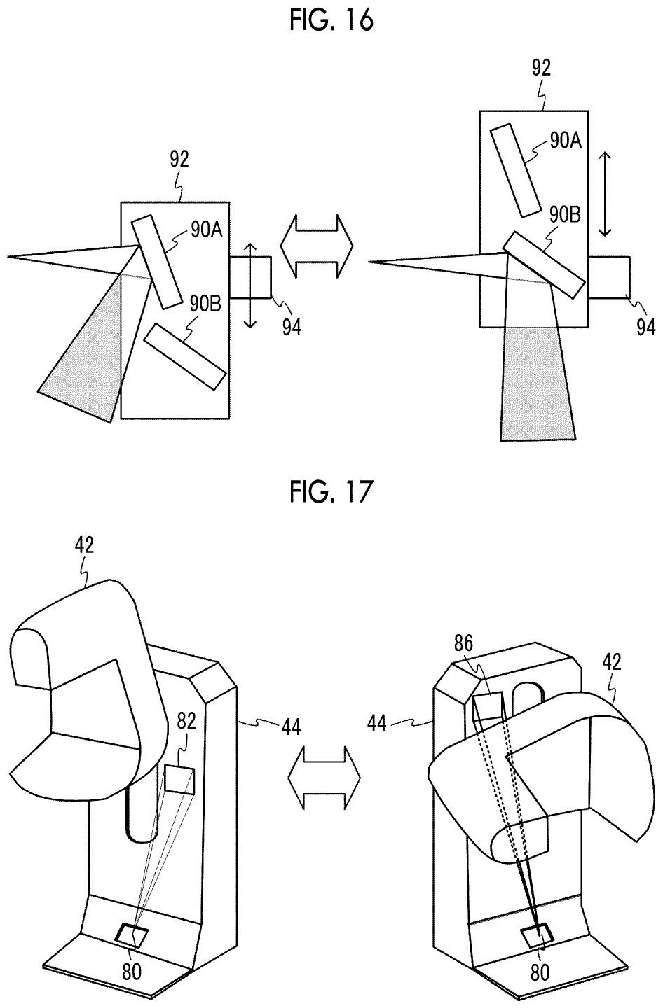

Alternatively, as illustrated in FIG. 16, an optical unit 92 including a plurality of reflective members 90A and 90B arranged to have different reflection directions may be provided, and the projection position may be switched by switching the reflective members 90A and 90B, which reflect the projection image, by driving a driving unit 94 including a motor or the like to move the optical unit 92, as illustrated in FIG. 16.

Alternatively, the projection position may be switched by changing each of the angle and the position of the reflective member 90.

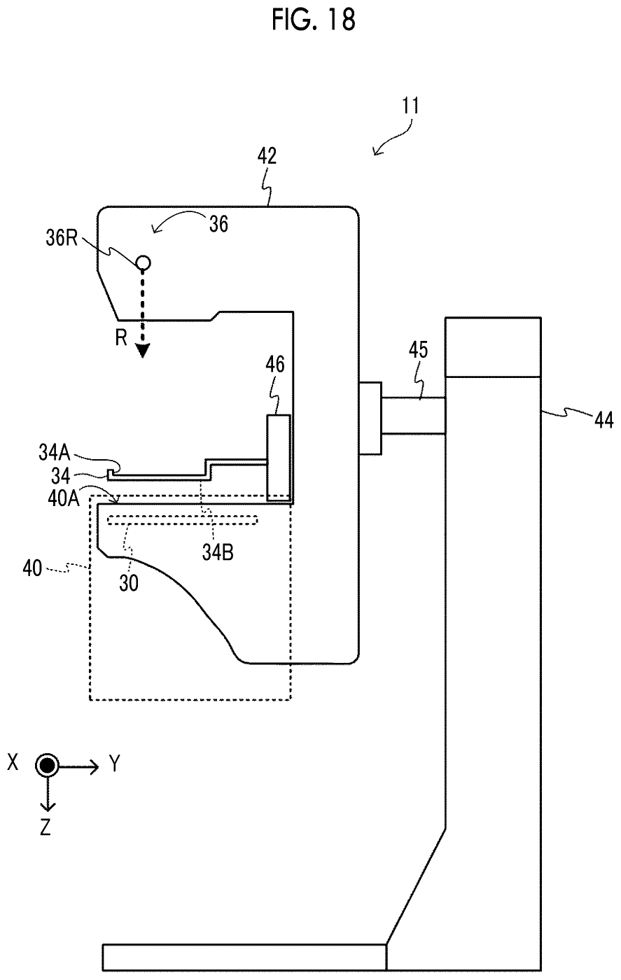

In addition, the projection unit 80, the first reflecting surface 82, and the second reflecting surface 86 may be provided at positions other than the arm part 42. For example, as illustrated in FIG. 17, the projection unit 80 may be provided on a floor portion of the lower side of the base 44, the first reflecting surface 82 may be provided in the vicinity of the center portion of the base 44, the second reflecting surface 86 may be provided in the vicinity of the upper portion of the base 44, and the reflecting surfaces where the projection is performed from the projection unit 80 may be switched according to the rotational position of the arm part 42.

Note that, in the embodiment described above, the ultrasound probe 70 is moved by the moving mechanism 72, but the moving mechanism 72 may be omitted, and the operator may manually operate the ultrasound probe 70. In addition, the biopsy unit 60 may be omitted. In a case where the biopsy unit 60 is omitted, the opening portion 34B of the bottom portion 34A of the compression member 34 is also unnecessary, and the operator operates the ultrasound probe 70 on the compression member 34 to acquire the ultrasound image. In a case where the opening portion 34B is omitted, it is desirable that the compression member 34 is formed of a material that easily propagates the ultrasound waves transmitted from the ultrasound probe 70. Examples of the material for the compression member 34 include resins such as polymethylpentene, polycarbonate, acrylic, or polyethylene terephthalate. In particular, polymethylpentene is suitable as the material for the compression member 34 since polymethylpentene has low rigidity, high elasticity, and high flexibility and has suitable values for acoustic impedance that affects the reflectivity of ultrasound waves and an attenuation coefficient that affects the attenuation of ultrasound waves. Note that the members that constitute the compression member 34 are not limited to the above. For example, the members that constitute the compression member 34 may be a film-like member. FIG. 18 illustrates an example of a mammography apparatus 11 in which the biopsy unit 60 and the moving mechanism 72 are omitted.

In the mammography apparatus 11 illustrated in FIG. 18, since the operator checks the ultrasound image while operating the ultrasound probe 70, the operator can check the ultrasound image displayed at a position where the ultrasound image is easy to see, by switching the display units according to the rotational position of the arm part 42 while operating the ultrasound probe 70.

In addition, a computer including various processors other than the CPU may execute various kinds of processing executed by the CPU executing software (program) in the embodiment described above. As the processors in this case, a programmable logic device (PLD) of which the circuit configuration can be changed after manufacture, such as a field-programmable gate array (FPGA), a dedicated electrical circuitry as a processor having a circuit configuration designed exclusively to execute specific processing, such as an application specific integrated circuit (ASIC), and the like are exemplified. In addition, the various kinds of processing may be executed by one of the various processors or may be executed by a combination of two or more processors of the same kind or different kinds (for example, a combination of a plurality of FPGAs and a combination of a CPU and an FPGA). In addition, the hardware structures of the various processors are more specifically electrical circuitry where circuit elements such as semiconductor elements are combined.

In addition, in the embodiment described above, the aspect in which various programs such as the display processing program are stored (installed) in the ROM 20B in advance has been described, but the disclosure is not limited thereto. Various programs may be provided in a form of being recorded in a recording medium such as a compact disk read only memory (CD-ROM), a digital versatile disk read only memory (DVD-ROM), and a Universal Serial Bus (USB) memory. In addition, the various programs may be provided in a form of being downloaded from an external information processing apparatus or the like via a network.

In addition, the configurations, operations, and the like of the mammography system 2, the mammography apparatus 10, the console 12, and the like described in the embodiment described above are examples, and may be changed according to a situation within a range not departing from the scope of the present disclosure.

The following supplementary notes are further disclosed with respect to the embodiment described above.

### Supplementary Note 1

A mammography apparatus comprising:
a support portion that rotatably supports a radiation source that emits radiation, at a plurality of imaging positions where incidence angles of the radiation to a breast of an examinee are different from each other;
a plurality of display units that display an ultrasound image of the breast acquired by an ultrasound probe, and are provided at different positions; and
at least one processor,
in which the processor performs processing of displaying the ultrasound image by switching between the plurality of display units according to a rotational position of the support portion.

### Supplementary Note 2

The mammography apparatus according to Supplementary Note 1,
in which in a case of switching between the plurality of display units according to the rotational position, in a case where there are the plurality of display units that can be visually recognized by an operator, the processor displays the ultrasound image by preferentially switching to the display unit close to the breast.

### Supplementary Note 3

The mammography apparatus according to Supplementary Note 1 or 2,
in which the plurality of display units are direct-viewing electronic displays.

### Supplementary Note 4

The mammography apparatus according to Supplementary Note 1 or 2,
in which the plurality of display units are projection-type electronic displays which display a projection image from a projection unit.

### Supplementary Note 5

The mammography apparatus according to Supplementary Note 4,
in which a support table that supports the support portion is provided with a reflecting surface functioning as the projection-type electronic display.

### Supplementary Note 6

The mammography apparatus according to Supplementary Note 4 or 5,
in which the projection unit has a reflective member, and switches between projection positions of the electronic display by changing at least one of an angle or a position of the reflective member.

### Supplementary Note 7

The mammography apparatus according to Supplementary Note 6,
in which a plurality of reflective members are provided.

### Supplementary Note 8

The mammography apparatus according to any one of Supplementary Notes 1 to 7, further comprising:
a detection unit that detects a rotation angle of the support portion,
in which in a case where the rotation angle equal to or greater than a preset threshold value is detected by the detection unit, the processor displays the ultrasound image by switching between the plurality of display units.

### Supplementary Note 9

The mammography apparatus according to any one of Supplementary Notes 1 to 7, further comprising:
a detection unit that detects a rotation angle of the support portion,
in which the processor displays the ultrasound image by switching to a predetermined display unit according to an angle detected by the detection unit, among the plurality of display units.

### Supplementary Note 10

A display method of a mammography apparatus, the display method comprising:
via a computer,
performing processing of displaying an ultrasound image of a breast acquired by an ultrasound probe, by switching between a plurality of display units, which display the ultrasound image and are provided at different positions, according to a rotational position of a support portion that rotatably supports a radiation source that emits radiation, at a plurality of imaging positions where incidence angles of the radiation to the breast of an examinee are different from each other.

### Supplementary Note 11

A display program of a mammography apparatus, the display program causing a computer to execute
processing of displaying an ultrasound image of a breast acquired by an ultrasound probe, by switching between a plurality of display units, which display the ultrasound image and are provided at different positions, according to a rotational position of a support portion that rotatably supports a radiation source that emits radiation, at a plurality of imaging positions where incidence angles of the radiation to the breast of an examinee are different from each other.

What is claimed is:

1. A mammography apparatus comprising:
a bracket that rotatably supports a radiation source that emits radiation, at a plurality of imaging positions where incidence angles of the radiation to a breast of an examinee are different from each other;
a plurality of displays that display an ultrasound image of the breast acquired by an ultrasound probe, and are provided at different positions; and
a processor,
wherein the processor performs processing of displaying the ultrasound image by switching between the plurality of displays according to a rotational position of the bracket.

2. The mammography apparatus according to claim 1,
wherein in a case of switching between the plurality of displays according to the rotational position, in a case where there are the plurality of displays that can be visually recognized by an operator, the processor displays the ultrasound image by preferentially switching to the display close to the breast.

3. The mammography apparatus according to claim 1,
wherein the plurality of displays are direct-viewing electronic displays.

4. The mammography apparatus according to claim 1,
wherein the plurality of displays are projection-type electronic displays which display a projection image from a projector.

5. The mammography apparatus according to claim 4,
wherein a support table that supports the bracket is provided with a reflecting surface functioning as the projection-type electronic display.

6. The mammography apparatus according to claim 4,
wherein the projector has a reflective member, and switches between projection positions of the electronic display by changing at least one of an angle or a position of the reflective member.

7. The mammography apparatus according to claim 6,
wherein a plurality of reflective members are provided.

8. The mammography apparatus according to claim 1, further comprising:
a detector that detects a rotation angle of the bracket,
wherein in a case where the rotation angle equal to or greater than a preset threshold value is detected by the detector, the processor displays the ultrasound image by switching between the plurality of displays.

9. The mammography apparatus according to claim 1, further comprising:
a detector that detects a rotation angle of the bracket,
wherein the processor displays the ultrasound image by switching to a predetermined display according to an angle detected by the detector, among the plurality of displays.

10. A display method of a mammography apparatus, the display method comprising:
via a computer,
performing processing of displaying an ultrasound image of a breast acquired by an ultrasound probe, by switching between a plurality of displays, which display the ultrasound image and are provided at different positions, according to a rotational position of a bracket that rotatably supports a radiation source that emits radiation, at a plurality of imaging positions where incidence angles of the radiation to the breast of an examinee are different from each other.

11. A non-transitory computer-readable storage medium storing a display program of a mammography apparatus, the display program causing a computer to execute
processing of displaying an ultrasound image of a breast acquired by an ultrasound probe, by switching between a plurality of displays, which display the ultrasound image and are provided at different positions, according to a rotational position of a bracket that rotatably supports a radiation source that emits radiation, at a plurality of imaging positions where incidence angles of the radiation to the breast of an examinee are different from each other.
